(19) United States
(12) Patent Application Publication
(10) Pub. No.: US 2007/0197781 A1
(43) Pub. Date: Aug. 23, 2007

(54) PROCESSES FOR THE PREPARATION OF CARBAPENEMS

(76) Inventors: Neera Tewari, Gurgaon (IN); Avinash Mane, Gurgaon (IN); Bishwa Prakash Rai, Azamgarh (IN); Mohan Prasad, Gurgaon (IN)

Correspondence Address: RANBAXY INC., 600 COLLEGE ROAD EAST, SUITE 2100, PRINCETON, NJ, 08540, US

(21) Appl. No.: 11/461,069

(22) Filed: Jul. 31, 2006

(30) Foreign Application Priority Data
Jul. 29, 2005 (IN) 2022/DEL/2005

(51) Int. Cl. C07D 487/04

(52) U.S. Cl. 540/350

(57) ABSTRACT

The invention relates to processes for the preparation of carbapenems. More particularly, it relates to a process for the preparation of meropenem.

### FIELD OF THE INVENTION

The field of the invention relates to processes for the preparation of carbapenems. More particularly, it relates to a process for the preparation of meropenem.

### BACKGROUND OF THE INVENTION

(4R,5S,6S)-3-[[(3S,5S)-5-(Dimethylcarbamoyl)-3-pyrrolidinyl]thio]-6-[(1R)-1-hydroxy-ethyl]-4-methyl-7-oxo-1-azabicyclo[3.2.0]hept-2-ene-2-carboxylic acid, commonly known as meropenem of Formula I is a synthetic, broad-spectrum, carbapenem antibiotic.

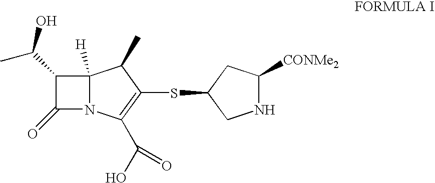

U.S. Pat. No 4,943,569 discloses a process for the preparation of meropenem, by reaction of enolphosphate of Formula II, 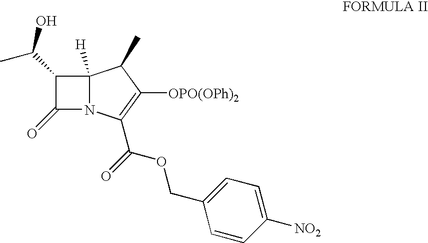
with a thiol side chain of Formula III in the presence of diisopropylethylamine, 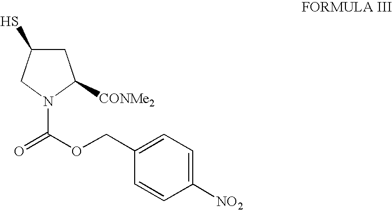
to provide a protected meropenem of Formula IV.

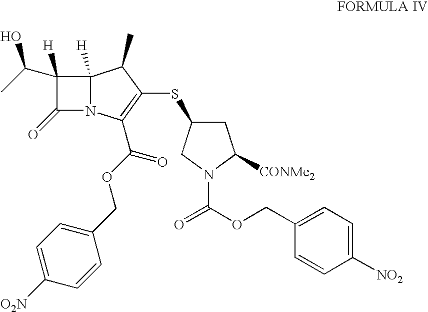

The compound of Formula IV is then deprotected by using palladium catalyst to get meropenem.

The thiol side chain of Formula III is prepared by S-deacylation of the compound of Formula V in the presence of aqueous sodium hydroxide and hydrochloric acid.

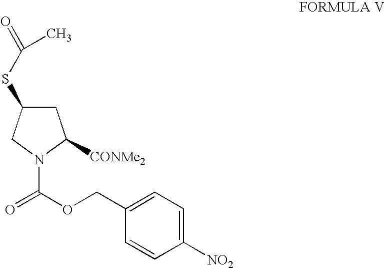

Similar processes for preparing meropenem have also been disclosed in U.S. Pat. Nos. 4,888,344 and 5,122,604; Sunagawa M., et al., J. Antibiot. (Tokyo), 1990, 43(5), 519-532 and Haruki M., et al., Heterocycles, 1995, 36, 145-159.

All the reported processes for the preparation of meropenem involve the isolation of the thiol side chain of Formula III, which in turn reacts with enol phosphate. Further, the preparation of thiol side chain of Formula III by S-deacylation involves a strong base such as sodium hydroxide. These processes also involve the isolation of the protected meropenem of Formula IV prior to deprotection.

### SUMMARY OF THE INVENTION

In one general aspect there is provided a process for the preparation of compound of Formula Ia, 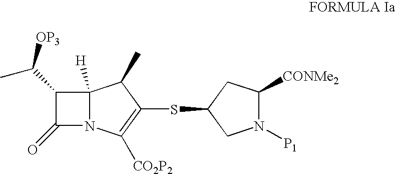
wherein P₁ represents hydrogen or an amino protecting group, P₂ represents hydrogen or a carboxyl protecting group and P₃ represents hydrogen or a hydroxyl protecting group. The process includes:

a) deprotecting thiol group of the compound of Formula Va,

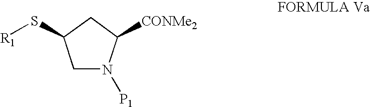

wherein P₁ is as defined above, R₁ is a thiol protecting group, to get a compound of formula IIIa,

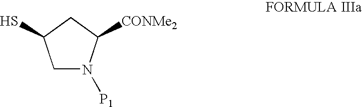

wherein P₁ is as defined above;

b) reacting the compound of Formula IIIa with a compound of Formula IIa,

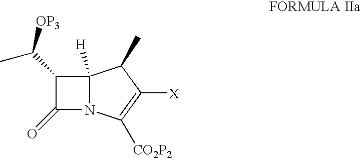

wherein P₂ and P₃ are as defined above and X represents OP(O)(OR)₂ or OSO₂R, wherein R represents substituted or unsubstituted C₁₋₆ alkyl, aralkyl or aryl, to get the compound of Formula Ia; and

c) isolating the compound of Formula Ia from the reaction mass thereof,

wherein the compound of formula IIIa is not isolated from the reaction mixture.

In another general aspect there is provided a process for the preparation of compound of Formula Ia, 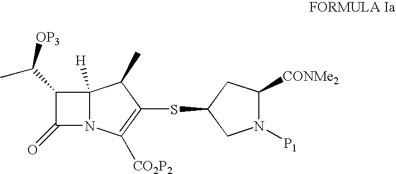
wherein P₁ represents hydrogen or an amino protecting group, P₂ represents hydrogen or a carboxyl protecting group and P₃ represents hydrogen or a hydroxyl protecting group.
The process includes:

a) treating compound of Formula Va with pyrrolidine,

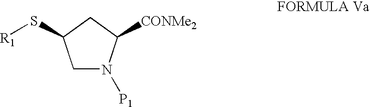

wherein P₁ is as defined above and R₁ is a thiol protecting group, to get compound of formula IIIa

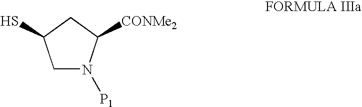

wherein P₁ is as defined above;

b) reacting the compound of Formula IIIa with a compound of Formula IIa,

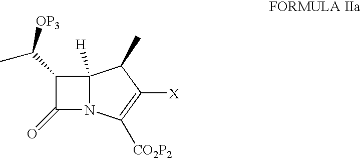

wherein P₂ and P₃ are as defined above and X represents OP(O)(OR)₂ or OSO₂R, wherein R represents substituted or unsubstituted C₁₋₆ alkyl, aralkyl or aryl, to get the compound of Formula Ia; and

c) isolating the compound of Formula Ia from the reaction mass thereof.

In another general aspect there is provided a process for the preparation of meropenem of Formula I. The process includes:

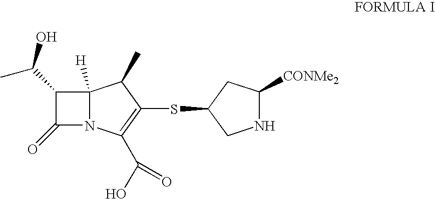

a) deprotecting thiol group of compound of Formula Vb,

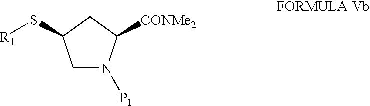

wherein P₁ is an amino protecting group and R₁ is a thiol protecting group, to get compound of formula IIIb,

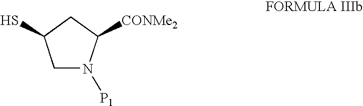

wherein P₁ is as defined above;

b) reacting the compound of Formula IIIb with a compound of Formula IIb,

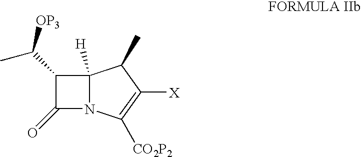

wherein P₂ is a carboxyl protecting group, P₃ is hydrogen or a hydroxyl protecting group and X represents OP(O)(OR)₂ or OSO₂R, wherein R represents substituted or unsubstituted C₁₋₆ alkyl, aralkyl or aryl, to get compound of Formula Ib,

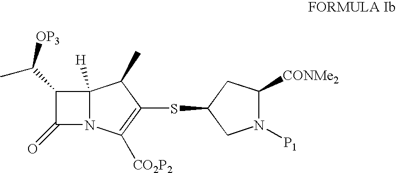

wherein P₁, P₂ and P₃ are as defined above;

c) deprotecting the compound of Formula Ib to get meropenem of Formula I; and

d) isolating the meropenem of Formula I from the reaction mass thereof,

wherein the compound of Formula Ib is not isolated from the reaction mixture.

The details of one or more embodiments of the inventions are set forth in the description below. Other features, objects and advantages of the inventions will be apparent from the description and claims.

### DETAILED DESCRIPTION OF THE INVENTION

The present inventors have developed a process for the preparation of meropenem and its analogues. The process does not involve the isolation of S-deprotected thiol side chain and the protected meropenem intermediate, thereby reducing the work-up time as well as the cost of production. The present inventors have also found that the S-deprotection of the thiol side chain can be carried out in the presence of pyrrolidine and eliminates the need of strong basic conditions. By following the present process, the yield and purity of the final product, meropenem, is also considerably improved.

The term “protecting group” in the present invention refers to those used in the art and serve the function of blocking the carboxyl, amino or hydroxyl groups while the reactions are carried out at other sites of the molecule. Examples of a carboxyl protecting group include, but not limited to, optionally substituted C₁-C₈ alkyl, optionally substituted C₃-C₈ alkenyl, optionally substituted C₇-C₁₉ aralkyl, optionally substituted C₆-C₁₂ aryl, optionally substituted C₁-C₁₂ amino, optionally substituted C₃-C₁₂ hydrocarbonated silyl, optionally substituted C₃-C₁₂ hydrocarbonated stannyl, and a pharmaceutically active ester forming group. Examples of hydroxyl and amino protecting groups include, but not limited to, lower alkylsilyl groups, lower alkoxymethyl groups, aralkyl groups, acyl groups, lower alkoxycarbonyl groups, alkenyloxycarbonyl groups and aralkyloxycarbonyl groups.

A first aspect of the present invention provides a process for the preparation of compound of Formula Ia, 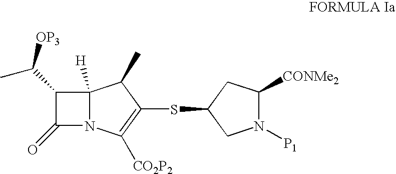
wherein P₁ represents hydrogen or an amino protecting group, P₂ represents hydrogen or a carboxyl protecting group and P₃ represents hydrogen or a hydroxyl protecting group, which comprises

a) deprotecting thiol group of compound of Formula Va,

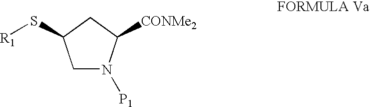

wherein P₁ is as defined above and R₁ is a thiol protecting group, to get compound of formula IIIa,

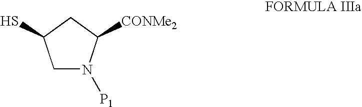

wherein P₁ is as defined above;

b) reacting the compound of Formula IIIa with a compound of Formula IIa,

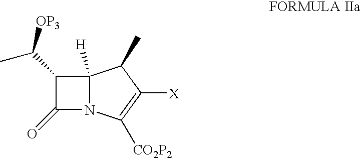

wherein P₂ and P₃ are as defined above and X represents OP(O)(OR)₂ or OSO₂R, wherein R represents substituted or unsubstituted C₁₋₆ alkyl, aralkyl or aryl, to get the compound of Formula Ia; and

c) isolating the compound of Formula Ia from the reaction mass thereof,

wherein the compound of formula IIIa is not isolated from the reaction mixture.

A second aspect of the present invention provides a process for the preparation of compound of Formula Ia, 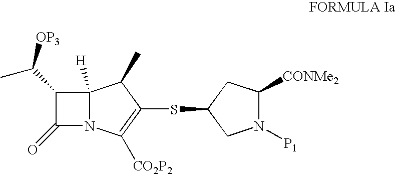
wherein P₁ represents hydrogen or an amino protecting group, P₂ represents hydrogen or a carboxyl protecting group and P₃ represents hydrogen or a hydroxyl protecting group, which comprises

a) treating compound of Formula Va with pyrrolidine,

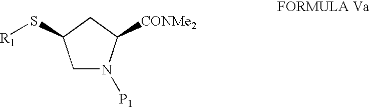

wherein P₁ is as defined above and R₁ is a thiol protecting group, to get compound of formula IIIa,

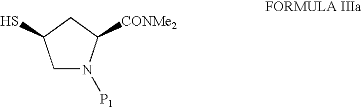

wherein P₁ is as defined above;

b) reacting the compound of Formula IIIa with a compound of Formula IIa,

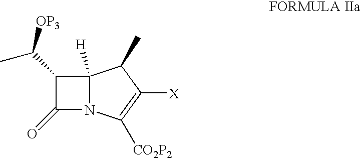

wherein P₂ and P₃ are as defined above and X represents OP(O)(OR)₂ or OSO₂R, wherein R represents substituted or unsubstituted C₁₋₆ alkyl, aralkyl or aryl, to get the compound of Formula Ia; and

c) isolating the compound of Formula Ia from the reaction mass thereof.

A third aspect of the present invention provides a process for the preparation of meropenem of Formula I,

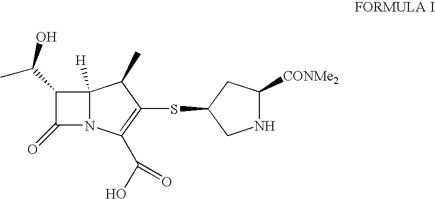

which comprises

a) deprotecting thiol group of compound of Formula Vb,

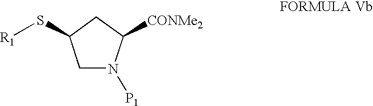

wherein P₁ is an amino protecting group and R₁ is a thiol protecting group, to get compound of formula IIIb,

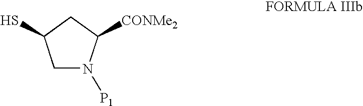

wherein P₁ is as defined above;

b) reacting the compound of Formula IIIb with a compound of Formula IIb,

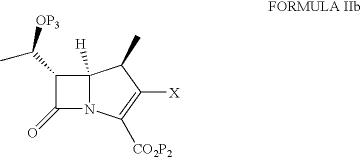

wherein P₂ is a carboxyl protecting group, P₃ is hydrogen or a hydroxyl protecting group and X represents OP(O)(OR)₂ or OSO₂R, wherein R represents substituted or unsubstituted C₁₋₆ alkyl, aralkyl or aryl, to get compound of Formula Ib,

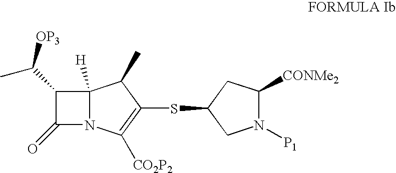

wherein P₁, P₂ and P₃ are as defined above;

c) deprotecting the compound of Formula Ib to get meropenem of Formula I; and

d) isolating the meropenem of Formula I from the reaction mass thereof,

wherein the compound of Formula Ib is not isolated from the reaction mixture.

Enol-phosphate of Formula Ia and thiol side chain of Formula Va can be prepared by processes reported in the prior-art as mentioned earlier. Thiol side chain is dissolved in an organic solvent and cooled to a temperature of about 25° C. or less. Pyrrolidine is added to the reaction mixture and stirred for a sufficient time to effect deprotection of the thiol group. The reaction mixture so obtained can optionally be treated with an aqueous mineral acid solution. Enolphosphate is added to the organic layer of the reaction mixture at a temperature of about 0° C. or less. The reaction mixture is stirred in the presence of a base for a sufficient time at the same temperature to effect the coupling reaction. The reaction mixture is subsequently hydrogenated using a palladium catalyst in the presence of a non nucleophilic buffer. Examples of buffers include morpholinopropanesulphonic acid and morpholinoethanesulphonic acid. An aqueous buffer comprising N-methylmorpholine mat also be used.

After completion of the reaction, the solid product is isolated from the aqueous layer, washed with an organic solvent and dried to get meropenem.

While the present invention has been described in terms of its specific embodiments, certain modifications and equivalents will be apparent to those skilled in the art and are intended to be included within the scope of the present invention.

### EXAMPLE 1

### Preparation of Meropenem

4-Nitrobenzyl (2S,4S)-4-(acetylthio)-2-[(dimethylamino)carbonyl]pyrrolidine-1-carboxylate (50 g) was dissolved in N,N-dimethylformamide (500 ml) and cooled to −5° to 0° C., followed by drop-wise addition of pyrrolidine (13.5 g) at the same temperature. The reaction mixture was stirred at −5° to 0° C. for 30 minutes and cooled to −40° to −35° C. 4-Nitrobenzyl (4R,5R,6S)-3-[(diphenoxyphosphoryl)oxy]-6-[(1R)-1-hydroxyethyl]-4-methyl-7-oxo-1-azabicyclo[3.2.0]hept-2-ene-2-carboxylate (Enolphosphate; 50 g) was added to the reaction mixture, followed by the addition of diisopropylethylamine (14.0 g) and stirring for 60 minutes at the same temperature. The reaction mixture was then poured into a mixture of ethyl acetate (500 ml) and water (500 ml). The ethyl acetate layer was separated and mixed with buffer containing N-methylmorpholine in water (500 ml) at a pH of 7.0. The hydrogenation of the reaction mixture so obtained was carried out at ambient temperature over palladium-carbon and the aqueous layer was separated after hydrogenation. Acetone (2 L) was added at 0°-5° C. to the aqueous layer of the reaction mixture to obtain the title compound in crystalline form.
Yield: 20 g
HPLC Purity: 98%

### EXAMPLE 2

### Preparation of Meropenem

4-Nitrobenzyl (2S,4S)-4-(acetylthio)-2-[(dimethylamino)carbonyl]pyrrolidine-1-carboxylate (30 g) was dissolved in dichloromethane (90 ml) and cooled to -10° to 0° C., followed by drop-wise addition of pyrrolidine (8.0 g) at the same temperature. The reaction mixture was stirred at −5° to 0° C. for 30 minutes and poured into 5% hydrochloric acid (150 ml), followed by separation of the organic layer. 4-Nitrobenzyl (4R,5R,6S)-3-[(diphenoxyphosphoryl)oxy]-6-[(1R)-1-hydroxyethyl]-4-methyl-7-oxo-1-azabicyclo[3.2.0]hept-2-ene-2-carboxylate (Enolphosphate; 30 g) was dissolved in dimethylformamide (150 ml) and cooled to −40° to −50° C., followed by the addition of dichloromethane solution containing 4-nitrobenzyl (2S,4S)-2-[(dimethylamino)carbonyl]-4-mercaptopyrrolidine-1-carboxylate. Diisopropylethylamine (8.4 g) was added drop-wise to the reaction mixture so obtained and stirred for 60 minutes at −40° to −30° C. The reaction mixture was then poured into a mixture of ethyl acetate (300 ml) and water (300 ml). The ethyl acetate layer was separated and hydrogenated at pH 7.0 over palladium-carbon and the aqueous layer was separated after hydrogenation, followed by treatment with activated carbon. The solution so obtained was filtered and acetone (2 L) was added at 0°-5° C. and stirred for 3 h at the same temperature. The reaction mixture was filtered, washed with acetone and dried to obtain the title compound.
Yield: 11 g
HPLC Purity: 98%

While the present invention has been described in terms of its specific embodiments, certain modifications and equivalents will be apparent to those skilled in the art and are intended to be included within the scope of the present invention.

We claim:

1. A process for the preparation of compound of Formula Ia, 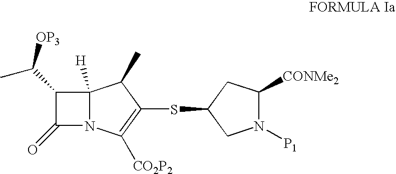
wherein P₁ represents hydrogen or an amino protecting group, P₂ represents hydrogen or a carboxyl protecting group and P₃ represents hydrogen or a hydroxyl protecting group, the process comprising:
a) deprotecting thiol group of compound of Formula Va, 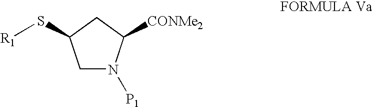
wherein P₁ is as defined above and R₁ is a thiol protecting group, to get compound of formula IIIa, 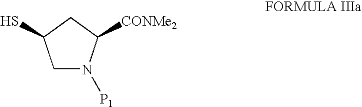
wherein P₁ is as defined above;
b) reacting the compound of Formula IIIa with a compound of Formula IIa, 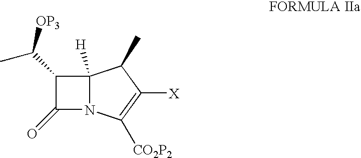
wherein P₂ and P₃ are as defined above and X represents OP(O)(OR)₂ or OSO₂R, wherein R represents substituted or unsubstituted C₁₋₆ alkyl, aralkyl or aryl, to get the compound of Formula Ia; and
c) isolating the compound of Formula Ia from the reaction mass thereof,
wherein the compound of formula IIIa is not isolated from the reaction mixture.

2. The process as claimed in claim 1, wherein step b) is carried out in the presence of an organic base.

3. The process as claimed in claim 2, wherein the organic base is diisopropylethylamine.

4. The process as claimed in claim 1, wherein step b) is carried out at a temperature of 0° C. or less.

5. The process as claimed in claim 1, wherein the thiol protecting group comprises acetyl and benzoyl.

6. A process for the preparation of compound of Formula Ia, 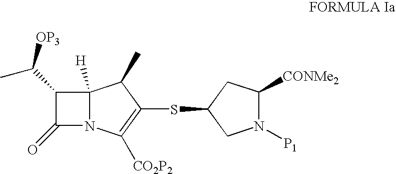
wherein P₁ represents hydrogen or an amino protecting group, P₂ represents hydrogen or a carboxyl protecting group and P₃ represents hydrogen or a hydroxyl protecting group the process comprising:
a) treating compound of Formula Va with pyrrolidine, 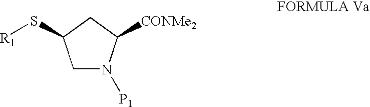
wherein P₁ is as defined above and R₁ is a thiol protecting group, to get compound of formula IIIa, 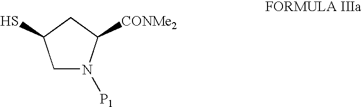
wherein P₁ is as defined above;
b) reacting the compound of Formula IIIa with a compound of Formula IIa, 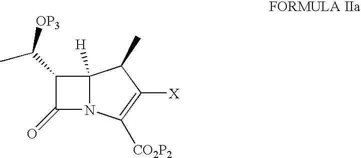
wherein P₂ and P₃ are as defined above and X represents OP(O)(OR)₂ or OSO₂R, wherein R represents substituted or unsubstituted C₁₋₆ alkyl, aralkyl or aryl, to get the compound of Formula Ia; and
c) isolating the compound of Formula Ia from the reaction mass thereof.

7. The process as claimed in claim 6, wherein step a) is carried out at a temperature of 25° C. or less.

8. The process as claimed in claim 6, wherein step a) comprises acidification of reaction mixture with a mineral acid.

9. The process as claimed in claim 6, wherein the thiol protecting group comprises acetyl and benzoyl.

10. A process for the preparation of meropenem of Formula I, 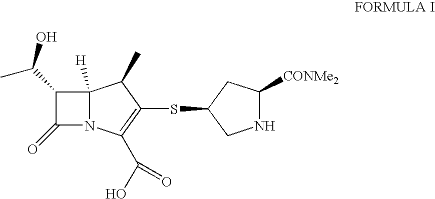
the process comprising:
a) deprotecting thiol group of compound of Formula Vb, 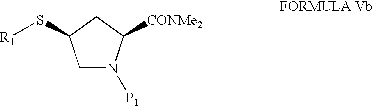
wherein P₁ is an amino protecting group, R₁ is a thiol protecting group, to get compound of formula IIIb, 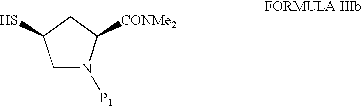
wherein P₁ is as defined above;
b) reacting the compound of Formula IIIb with a compound of Formula IIb, 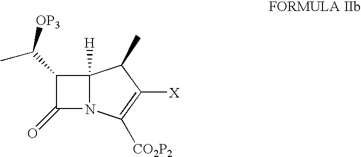
wherein P₂ is a carboxyl protecting group, P₃ is hydrogen or a hydroxyl protecting group and X represents OP(O)(OR)₂ or OSO₂R, wherein R represents substituted or unsubstituted C₁₋₆ alkyl, aralkyl or aryl, to get compound of Formula Ib, 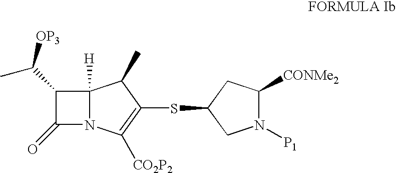
wherein P₁, P₂ and P₃ are as defined above;
c) deprotecting the compound of Formula Ib to get meropenem of Formula I; and
d) isolating the meropenem of Formula I from the reaction mass thereof,
wherein the compound of Formula Ib is not isolated from the reaction mixture.

11. The process as claimed in claim 10, wherein the thiol protecting group comprises acetyl and benzoyl.

12. The process as claimed in claim 10, wherein step c) is carried out in the presence of a palladium catalyst.

13. The process as claimed in claim 10, wherein step c) is carried out in the presence of an aqueous buffer.

14. The process as claimed in claim 10, wherein step c) is carried out in the presence of a non-nucleophilic buffer and in biphasic solvent system.

15. The process as claimed in claim 14, wherein the non-nucleophilic buffer comprises morpholinopropanesulphonic acid and morpholinoethanesulphonic acid.
